(19) United States
(12) Patent Application Publication
(10) Pub. No.: US 2013/0184354 A1
(43) Pub. Date: Jul. 18, 2013

(54) Silicone and Hylauronic Acid (HLA) Delivery Systems for Products by Sustainable Processes for Medical Uses Including Wound Management

(71) Applicants: Donna K. Jackson, Forth Worth, TX (US); Keith Hyatt, Anaheim, CA (US)

(72) Inventors: Donna K. Jackson, Forth Worth, TX (US); Keith Hyatt, Anaheim, CA (US)

(21) Appl. No.: 13/739,808

(22) Filed: Jan. 11, 2013

Related U.S. Application Data
(60) Provisional application No. 61/586,693, filed on Jan. 13, 2012.

(51) Int. Cl. A61K 47/36; A61K 47/34

(52) U.S. Cl. CPC A61K 47/36; A61K 47/34
USPC 514/729

(57) ABSTRACT

Topically absorbable compositions with bioactive or bioavailable cannabis-derived cannabinoids known to be effective for CB1 or CB2 modulation, and a plurality of indications for patients in need, and methods for producing composition, without detectable or biologically active levels of THC. Method uses a heat cycle process to combine cannabinoids with flax seed oil and at least one triglyceride, to produce an extract. The extract can include non-psychoactive agents, namely, CBD and accordingly does not deleteriously impact federal legal schemes or bone fide drug-testing regimes.

### STATEMENT OF PRIORITY AND CROSS-REFERENCE TO RELATED APPLICATIONS UNDER THE PARIS CONVENTION

The instant filing requesting grant of Letters Patent claims full Paris Convention Priority from U.S. Provisional Patent Applications Ser. No. 61/586,693, filed Jan. 13, 2012, name of the present inventor, and which is expressly incorporated herein by reference as if fully set forth herein.

### FIELD OF THE DISCLOSURE

The ability to combine the ingredients derived from cannabinoids with silicon fluids, coupled with hyaluronic acid (HLA), can enhance current methods of applications and revolutionize the opportunity for absorption into the skin to help ease pain. Cellular membrane penetration appears to be among the working mechanisms in play, within the subject formulations initially reduced to practice and subsequent species, e.g., GJ® formulae, extract, or juice, referred to co-extensively throughout.

The current ingredients for GJ formulae that contains THC extracted from marijuana contains certain oils and is used to support and ease muscle pain as well as nerve pain. What is provided are ingredients and compositions with cannabidiol (CBD), that lack psychoactvitity.

The proposal to extend the teachings include various types of silicon liquids and silicon gels, for example, those being used in cosmetic applications along with hyaluronic acid (HLA). Silicone fluids have been used by the cosmetic industry to promote healing, while hyaluronic acid has been used by the cosmetic industry as an absorbing agent. The present disclosure provides silicone fluids as well as HLA, for example, for promoting healing and for enhanced absorption. The disclosure also provides silicone fluids that include antimicrobial solutions.

The present invention relates to enhanced products by sustainable processes for medicinal use. In particular, the present invention relates to newly formulated and stabilized products for medicinal use, for example, those offered for consideration under the auspices of Alaska Ballot Measure 8, California Proposition 215, Colorado Amendment 20, District of Columbia Amendment 618-622, Hawaii Senate Bill 862, Maine Ballot Question 2, Michigan Proposal 1, Montana Initiative 1448, Nevada Ballot Question 9, New Jersey Senate Bill 119, New Mexico Senate Bill 523, Oregon Ballot Measure 67, Rhode Island Senate Bill 0710, Vermont Senate Bill 76 HB 645, Washington Initiative 692, and related international, national, territorial, or later enacted statutory or legislative authority.

In order to overcome longstanding needs to provide alternative medicine sources to patients in need of the same, the present inventor has researched both historical and technical solutions to these issues. A detailed review of the literature failed to reveal any disclosure teaching what has been disclosed by the present inventor, in terms of profiling, compounding and delivering objects of the present invention. For this reason, and as set forth and claimed below, the utilities of the present solution, as offered for consideration herein are respectfully proposed to constitute invention, as defined by statute.

From Mesopotamian times until now, healers and shamans have recognized properties of elements derived from the Cannabis plant that are helpful to patients in need. Detailed mechanisms proposed for the same have been set forth below, as derived from the extensive literature surveyed along with historical and technical records. What is now patent is that selected use of the bioactive components of said plant, or cannabinoids, when managed properly, can be safely and effectively deployed medicinally, both with and without psychoactive effects. The present inventor has developed extracts allowing (what are believed to be naturally competing) levels of cannabinoids to be administered by moderating levels of THC (Tetrahydrocannabinol) relative to CBD (cannabidiol) and CBN (cannabinol) levels.

In essence, contrary to prior art collected and studied, the present inventions selectively isolate non-psychoactive cannabinoids so that they can be delivered to, and safely administered to, those in need. By addressing this longstanding need, to bifurcate psychoactive effects from other helpful properties and create medicinal products has not been fully addressed prior to the advent of the instant teachings. Likewise, no literature suggests or motivates those skilled in the art to undertake applicant's unique formulational approach, let alone prior art, therefore making the instant teachings progress in science and the useful arts, it is respectfully proposed, as fully disclosed herein, and claimed below.

By way of example, it is understood and believed that by chemically profiling different strains, phenotypes and expressed genetic variations can be selectively combined, hybridized, or otherwise selected to control the levels of various cannabinoids and their natural or inherent ratios managed, revised or altered as required. Combining strain profiling with user needs' profiling and database management is the interface between compassionate use and personalized medicine. Monitoring just three main cannabinoids; THC, CBD and CBN, can bridge the phenomenological gaps which exist with respectively different and genetically pre-selected strains, families and groupings. It is finally respectfully proposed that allowing patients to have full access to data about THC/CBD/CBN levels can substantially enhance their ability to make efficacious choices about the correct form and type of medicinal treatment which they choose.

### BACKGROUND OF CANNABINOIDS AND CANNABINOID RECEPTORS

Cannabinoid receptors are a class of cell membrane receptors under the G protein-coupled receptor superfamily. CB1 and CB2 are known cannabinoid receptors. Cannabinoid receptors are activated by three major group of ligands; endocannabinoids (known to be produced by the mammalian body), plant cannabinoids (such as THC, produced by the Cannabis plant) and synthetic cannabinoids (such as HU-210). All of the endocannabinoids and plant cannabinoids are lipophilic, i.e. fat soluble, compounds. Tetrahydrocannabinol (THC), cannabidiol (CBD) and cannabinol (CBN) are the most prevalent natural cannabinoids and have received the most study. Some other common cannabinoids are listed in U.S. Pat. No. 7,179,800, expressly incorporated herein by reference, as if fully set forth herein.

Phytocannabinoids are compounds found in the Cannabis plant that are structurally related to tetrahydrocannabinol (THC). Cannabis plants can exhibit wide variation in the quantity and type of cannabinoids they produce. The mixture of cannabinoids produced by a plant is known as the plant's cannabinoid profile. Selective breeding has been used to control the genetics of plants and modify the cannabinoid profile. Cannabis plants used as fiber (commonly called hemp) are bred for fiber and are known to have low levels of psychoactive chemicals like THC. Cannabis may also be selectively bred for high CBD content. However, prior to the advent of the instant teachings, no formulational approaches have been known to do this.

Plant or Phytocannabinoids, also called natural cannabinoids, herbal cannabinoids, and classical cannabinoids, are only known to occur naturally in significant quantity in the cannabis plant, and are concentrated in a viscous resin that is produced in glandular structures known as trichomes. In addition to cannabinoids, the resin is rich in terpenes, which are largely responsible for the odour of the cannabis plant. Some cannabinoids are described in U.S. Pat. No. 5,227,537, incorporated by reference expressly, as if fully set forth herein. Other identified cannabinoids are disclosed in Agurell et al (1986) Pharmacological Review, 38:31-43. Phytocannabinoids are nearly insoluble in water but are soluble in lipids, alcohols, and other non-polar organic solvents. However, as phenols, they form more water-soluble phenolate salts under strongly alkaline conditions. At least 100 cannabinoids have been isolated from the cannabis plant. Tetrahydrocannabinol (THC), cannabidiol (CBD) and cannabinol (CBN) are the most prevalent natural cannabinoids and have received the most study.

Endocannabinoids, found in the nervous and immune systems of animals and that activate cannabinoid receptors, are substances produced from within the body that activate cannabinoid receptors.

Synthetic cannabinoids encompass a variety of distinct chemical classes: the classical cannabinoids structurally related to THC, the non-classical cannabinoids including the aminoalkylindoles, 1,5-diarylpyrazoles, quinolines and arylsulphonamides, as well as eicosanoids related to the endocannabinoids.

It is well known that cannabinoids bind reversibly and stereo-selectively to the cannabinoid receptors. The affinity of an individual cannabinoid to each receptor determines the effect of that cannabinoid (British Journal of Pharmacology (2003) 138:767-774).

A correlation between inflammation in both the peripheral nervous system and in the central nervous systems (CNS) has been found with respect to some cannabinoid receptors such as CB2 (British Journal of Pharmacology (2008) 153:277-285).

Cannabinoid receptor agonists, may be useful in the treatment of pain, inflammation and autoimmune diseases. An agonist is a chemical that binds to a receptor of a cell and triggers a response by that cell. Agonists often mimic the action of a naturally occurring substance.

Whereas an agonist causes an action, an antagonist blocks the action of the agonist. A receptor antagonist is a type of receptor ligand, or drug that does not provoke a biological response itself upon binding to a receptor, but blocks or dampens agonist-mediated responses. In pharmacology, antagonists have affinity but no efficacy for their cognate receptors, and binding will disrupt the interaction and inhibit the function of an agonist or inverse agonist at receptors. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist receptor binding. The majority of drug antagonists achieve their potency by competing with endogenous ligands or substrates at structurally-defined binding sites on receptors.

All-natural cannabinoids are derived from their respective 2-carboxylic acids (2-COON) by decarboxylation (catalyzed by heat, light, or alkaline conditions).

Tetrahydrocannabinol (THC) is the primary psychoactive component of the plant. It has been shown to ease moderate pain (analgesic) and to be neuroprotective. THC has approximately equal affinity for the CB1 and CB2 receptors. Although years of selective breeding have sought to maximize THC levels, it is readily possible to down-modulate THC levels, as may be needed for many patients. However, no prior art teachings were developed which use flaxseed oil and Cannabis material, or ganja (defined herein as including the entire plant matter ground-up, per cultural traditions including certain secular authorities).

Ganja is the historical term which refers to the sum total of all plant parts.

Cannabidiol (CBD) is a cannabinoid found in Cannabis. It is a major constituent of the plant, representing up to 40% in its extracts (Grlic, L. (1976). “A Comparative Study on Some Chemical and Biological Characteristics of Various Samples of Cannabis Resin.” U.N. Bulletin on Narcotics 14: 37-46).

It has displayed sedative effects in animal tests (Pickens (1981) Sedative Activity of Cannabis in Relation to its Delta'-trans-tetrahydrocannabinol and Cannabidiol content. British Journal of Pharmacology, 72:649-656). Some research, however, indicates that CBD can increase alertness [Nicholson, A.; C. Turner, B. Stone, and P. Robson (2004). Effect of Delta-9-tetrahydrocannabinol and Cannabidiol on Nocturnal Sleep and Early-morning Behavior in Young Adults, Journal of Clinical Psychopharmacology 24:305-313). It may decrease the rate of THC clearance from the body, perhaps by interfering with the metabolism of THC in the liver. Cannabis indica strains often feature higher CBD content.

Medically, it has been shown to relieve convulsion, inflammation, anxiety, and nausea, as well as inhibit cancer cell growth (Mechoulam et al (2007) Cannabidiol - Recent Advances, Chemistry & Biodiversity 4:1678-1692). Recent studies have shown cannabidiol to be as effective as atypical antipsychotics in treating schizophrenia; Zuardi et al (2006) Cannabidiol, a Cannabis sativa Constituent, as an Antipsychotic Drug, Brazilian Journal of Medical & Biological Research, 39:421-429). Studies have also shown that it may relieve symptoms of dystonia (Consroe et al (1986) Open Label Evaluation of Cannabidiol in Dystonic Movement Disorders, International Journal of Neuroscience, 30: 277-282; Snider et al (1985) Beneficial and Adverse Effects of Cannabidiol in a Parkinson Patient with Sinemet-Induced Dystonic Dyskinesia, Neurology, (Suppl. 1) p. 201).

Cannabidiol has no affinity for CB1 and CB2 receptors but acts as an indirect antagonist of cannabinoid agonists (Mechoulam et al (2007). Cannabidiol - Recent Advances, Chemistry & Biodiversity 4:1678-1692). Recently it was found to be an antagonist at the putative new cannabinoid receptor, GPR55, a GPCR expressed in the caudate nucleus and putamen (Ryberg et al (2007) The Orphan Receptor GPR55 is a Novel Cannabinoid Receptor, British J. Pharmacology 152 :1092). Cannabidiol has also been shown to act as a 5-HT1A receptor agonist (Russo et al (2005) Agonistic properties of cannabidiol at 5-HT1a receptors, Neurochemical Research 30 (8): 1037-43.] an action which is involved in its antidepressant (Zanelati et al (2009) Antidepressant-like Effects of Cannabidiol in Mice: Possible Involvement of 5-HT Receptors, British Journal of Pharmacology 159:122-128; Resstel et al (2009) 5-HT1A Receptors are Involved in the Cannabidiol-induced Attenuation of Behavioral and Cardiovascular Responses to Acute Restraint Stress in Rats, British Journal of Pharmacology 156:181-188), anxiolytic (Campos et al (2008) Involvement of 5HT1A Receptors in the Anxiolytic-like effects of Cannabidiol Injected into the Dorsolateral Periaqueductal Gray of Rats, Psychopharmacology 199 :223-230) and neuroprotective effects (Mishima et al (2005) Cannabidiol Prevents Cerebral Infarction via a Serotonergic 5-hydroxytryptamine1A Receptor-dependent Mechanism, Stroke: A Journal of Cerebral Circulation 365:1077-1082; Hayakawa et al (2007) Repeated treatment with cannabidiol but not Delta-9-tetrahydrocannabinol has a neuroprotective effect without the development of tolerance, Neuropharmacology 52:1079-1087).

Cannabidiol has also been shown to inhibit cancer cell growth with low potency in non-cancer cells. Although the inhibitory mechanism is not yet fully understood, Ligresti et al. suggest that “cannabidiol exerts its effects on these cells through a combination of mechanisms that include either direct or indirect activation of CB2 and TRPV1 receptors, and induction of oxidative stress, all contributing to induce apoptosis (Ligresti et al (2006) Antitumor Activity of Plant Cannabinoids with Emphasis on the Effect of Cannabidiol on Human Breast Carcinoma, Journal of Pharmacology & Experimental Therapeutics, 318:1375-1387). It has also been reported that CBD shows promise for controlling the spread of metastatic breast cancer. In vitro CBD downregulates the activity of the gene ID1 which is responsible for tumor metastasis (McAllister et al (2007) Cannabidiol as a Novel Inhibitor of Id-1 Gene Expression in Aggressive Breast Cancer Cells, Molecular Cancer Therapeutics 6: 2921-2927).

Cannabinoids are traditionally separated from the plant by extraction with organic solvents. Hydrocarbons and alcohols are often used as solvents. However, these solvents are flammable and many are toxic. Supercritical solvent extraction with carbon dioxide is an alternative technique. Although this process requires high pressures (73 atmospheres or more), there is minimal risk of fire or toxicity, solvent removal is simple and efficient, and extract quality can be well-controlled. Once extracted, cannabinoid blends can be separated into individual components using wiped film vacuum distillation or other distillation techniques. However, to produce high purity cannabinoids, chemical synthesis or semisynthesis is generally required.

Medications containing natural or synthetic cannabinoids or cannabinoid analogs are:

Dronabinol (Marinol), is Δ9-tetrahydrocannabinol (THC), used as an appetite stimulant, anti-emetic, and analgesic

Nabilone (Cesamet), a synthetic cannabinoid and an analog of Marinol. It is Schedule II unlike Marinol, which is Schedule III

Sativex, a cannabinoid extract oral spray containing THC, CBD, and other cannabinoids used for neuropathic pain and spasticity in 22 countries including England, Canada and Spain. Sativex develops whole-plant cannabinoid medicines.

Rimonabant (SR141716), a selective cannabinoid (CB1) receptor antagonist used as an anti-obesity drug under the proprietary name Acomplia. It is also used for smoking cessation.

Other synthetic cannabinoids include:

JWH-018, a potent synthetic THC analogue discovered by Dr. John W. Huffman at Clemson University. It is being increasingly sold in legal smoke blends collectively known as “spice”. Several countries and states have moved to ban it legally

CP-55940, produced in 1974, this synthetic cannabinoid receptor agonist is many times more potent than THC

Dimethylheptylpyran

HU-210, about 100 times as potent as THC[28]

HU-331 a potential anti-cancer drug derived from cannabidiol that specifically inhibits topoisomerase II

SR144528, a CB2 receptor antagonists

WIN 55,212-2, a potent cannabinoid receptor agonist

JWH-133, a potent selective CB2 receptor agonist

Levonantradol (Nantrodolum), an anti-emetic and analgesic but not currently in use in medicine

### SUMMARY OF THE DISCLOSURE

Briefly stated, the disclosure provides topically absorbable compositions with bioactive or bioavailable cannabis-derived cannabinoids known to be effective for CB1 or CB2 modulation, and a plurality of indications for patients in need, and methods for producing composition, without detectable or biologically active levels of THC. Method uses a heat cycle process to combine cannabinoids with flax seed oil and at least one triglyceride, to produce an extract. The extract can include non-psychoactive agents, namely, CBD and accordingly does not deleteriously impact federal legal schemes or bone fide drug-testing regimes.

Briefly stated, the present disclosure provides a process for producing a cannabinoid and triglyceride composition, the process comprising: adding at least about one gallon of cold pressed flax seed oil having with a 214 degrees F. burn point to a container which can be covered; adding at least about 228 grams of ground up plant materials per about one gallon from material derived from cannabis plants; covering for between about 8 and up to 12 hours the container and cycling back and forth between a maximum heat of less that 214 degrees F. and a minimum heat of between at least about 195-198 degrees F. while mechanically agitating and alternating between maximum and minimum heat; cooling a resulting admixture down to room temperature; separating the plant material from the liquid wherein the liquid forms a flax seed oil and cannabinoids extract (F-C extract); and combining the same with one or both of a silicone liquid carrier and hyaluronic acid (HLA) carrier.

In another aspect, the disclosure provides the above process, and composition made by the above process, wherein the cycle times are between 90 and 120 minutes for each maximum heat and each minimum heat cycle; and, the duration of the mechanical agitation is between at least about 30 and about 148 seconds. Moroever, what is provided is the above process, and composition made by the process, further comprising the step of: placing said plant material within a filtering divider material which both contains the plant material and reduces the flow of plant materials out of the filtering divider material. Also embraced, is the above process, and composition made by the process, wherein the cannabis plants comprise cannabidiol (CBD) and delta(9)-tetrahydrocannabinol (THC), and wherein the ratio of [cannabidiol (CBD) (weight)]/[delta(9)-tetrahydrocannabinol (THC) (weight)] in the F-C extract is greater than 20/1 or is greater than 30/1. Further, what is envisioned is the above process, and composition made by the above process, further comprising separating aliquots of the combined F-C extract and carrier into single doses by volume, and placing each aliquot into a separate container. Also provided, is the above composition, wherein each aliquot has a volume of 0.05 mL to 0.50 mL.

In another aspect, what is provided is a composition prepared by the above process, further comprising one or more of silicone beads, an inhalable carrier, hyaluronic acid (HLA), a fatty acid, and a fatty acid derivative. Also, what is embraced is the above composition, further comprising an ingestible carrier that is a beverage, baked good, sauce, or dressing. Moreover, the disclosure encompasses the above composition prepared by the above process, that further comprises a cosmetic or lotion.

In a Ganja Juice® composition, what is provided is the above composition, comprising a topical carrier that contains: water; goat's milk; Hellanthus annus oil; soya glycine; theobroma cacao seed butter; butyospernum; park fruit; vegetable glycerin; glycol copolymer; isopropyl myristate; monostearate; acrylamide; octyldodecanol; butylcarbamate; palmitate PEG-320; idopropanol; polysorbate; ceterate-14; aloe vera extract; glyceril stearate; carbopol; essential oils, hyaluronic acid, and silicone oil.

In other composition embodiments, the disclosure provides composition prepared by the above method, wherein the ratio of cannabidiol (CBD) to delta(9)-tetrahydrocannabinol (THC) is reduced, when compared to the ratio of cannabidiol (CBD) to delta(9)-tetrahydrocannabinol (THC) in the non-processed ground up plant materials. Also provided is above composition, wherein the ratio of cannabidiol (CBD) to delta(9)-tetrahydrocannabinol (THC) that is reduced, results in lower or substantially de minimus psychoactive effects, when compared to those produced by a composition that is the non-processed ground up plant materials.

Also provided is a method for administering a substitute for medicinal marijuana to mammals, wherein the substitute for medical marijuana is a substance that comprises a composition prepared by the above method, the method comprising administering to a patient a substance that comprises the composition prepared by the above method.

In other composition embodiments, what is provided is a composition configured for topical application, comprising cannabidiol (CBD), hyaluronic acid, water, at least one fatty acid or fatty acid derivative, wherein the composition is a clear solution or is an emulsion when stored at 23 degrees C., and wherein the composition does not form a biphasic solution when stored at 23 degrees C. Also provided is the above composition, wherein the at least one fatty acid or fatty acid derivative that comprises isopropylmyristate, stearate, palmitic acid, lecithin, olive oil, or flax seed oil. Moreover, what is provided is the above composition, wherein the ratio (weight/weight) of cannabidiol (CBD) to delta(9)-tetrahydrocannabinol (THC), is greater than 10.0/1.0, or is greater than 30.0/1.0.

In yet another composition embodiment, what is provided is a composition configured for topical or oral administration, wherein the composition is derived from cannabis plants that contain cannabidiol (CBD) and delta(9)-tetrahydrocannabinol (THC), wherein the ratio (weight/weight) of cannabidiol (CBD) to delta(9)-tetrahydrocannabinol (THC) in the composition is greater than 20/1, or is greater than 30/1, and wherein the composition comprises one or both of a silicone liquid and hyaluronic acid (HLA) carrier. Also, what is provided is above composition, further comprising at least one fatty acid or fatty acid derivative that comprises isopropylmyristate, stearate, palmitic acid, lecithin, olive oil, or flax seed oil. Also provided is above composition, that comprises silicone liquid and does not comprise hyaluronic acid. Also provided is above composition, that comprises hyaluronic acid and does not comprise silicone liquid.

What is provided is a process for producing a composition with bioactive and/or bioavailable Cannabis-derived cannabinoids known to be effective for CB1 and/or CB2 modulation, and a plurality of indications for patients in need. Using a heat cycle process to combine cannabinoids, including but not limited to THC and CBD with flax seed oil and at least one of the triglycerides therein, an extract is formulated which enables substantially profiled and Cannabinoid ratio-balanced aliquots (“miquots”) to be offered for consideration to patients, including non-psychoactive topically and orally delivered products and systems.

According to embodiments, there is provided a process for producing a cannabinoid and triglyceride composition; the process comprising, in combination; adding at least about one gallon of cold pressed flax seed oil having with a 214 F burn point to a container which can be covered; adding at least about 228 grams of ground up plant materials per about one gallon from material derived from Cannabis plants; covering for between about 8 and up to12 hours the container and cycling back and forth between a maximum heat of less that 214 degrees F. and a minimum heat of between about 195-198 F while mechanically agitating while alternating between maximum and minimum heat; cool a resulting admixture down to room temperature; and, separating the plant material from the liquid wherein the liquid forms an F-C extract.

According to embodiments, there is provided a method for administering medicinal marijuana to mammals, comprising, in combination; profiling Cannabinoid levels in Cannabis plant material, extracting a pharmaceutically effective dose, and combining the same with a carrier, namely, silicon-based or derived oils.

Broadly stated, the instant teachings provide a process for producing a composition with bioactive and/or bioavailable CB1 and/or CB2 modulation. Using a heat cycle process to combine cannabinoids, including but not limited to THC and CBD with flax seed oil and at least one of the triglycerides therein, an extract is formulated which enables substantially profiled and Cannabinoid ratio-balanced aliquots to be offered for consideration to patients, including non-psychoactive topically and orally delivered products and systems.

What is provided is above method, and compositions made by above method, that comprise avocado oil. What is provided is above composition, wherein the same does not deleteriously impact federal legal schemes or bone fide THC-based drug-testing regimes. Federal laws regulating marijuana are available (see, e.g., Garvey, T. (2012) Medical Marijuana: The Supremacy clause, Federalism, and the Interplay Between State and Federal Laws, CRS Report for Congress).

### DETAILED DISCLOSURE

The present disclosure provides methods and compositions produced therefrom, that mix silicon liquid along with HLA into Ganja Juice® (GJ) ingredients, either eliminating or enhancing those ingredients currently being used. Combination can also be mixed directly into the existing patent pending process of the filamentous fibrous spun silicon currently being held by DKJ silicone base matrix (“DJ sbm”).

Ganja Juice® is a proprietary formulation, available from Truly Hemp, 1313 North Miller Avenue, Anaheim, Calif., to licensed care-givers and those statutorily empowered to acquire the same) contains the following ingredients: water; goat's milk; Hellanthus annus (sunflower) oil; soya glycine; theobroma cacao seed butter; butyospernum; park fruit; vegetable glycerin; glycol copolymer; isopropyl myristate; monostearate; acrylamide; octyldodecanol; butylcarbamate; palmitate PEG-320; idopropanol; polysorbate; ceterate-14; aloe vera extract; glyceril stearate; carbopol; essential oils, hyaluronic acid, and silicone oils.

Ganja is the historical term which refers to the sum total of all plant parts. Sustainable agriculture involves making use of all offered for consideration. Not unlike the Native Americans' approach to preserving and harvesting each precious morsel in a sacrificed bison, the process of the present invention wastes no remnants.

In another aspect, the present disclosure provides a composition that comprises: (1) hyaluronic acid; (2) fatty acid; (3) water and goat's milk; (4) water, goat's milk, and Hellanthus annus oil; (5) water, goat's milk, Hellanthus annus oil, and soya glycine; (6) water, goat's milk, Hellanthus annus oil, soya glycine, and theobroma cacao seed butter; (7) water, goat's milk, Hellanthus annus oil, soya glycine, and theobroma cacao seed butter, and butyospernum; or any other combination of the ingredients of Ganja Juice®. For example, what is also provided is a composition, and related methods, that comprise: (8) water, Hellanthus annus oil, and isopropyl myristate, or for example, (9) water, butyospernum, and polysorbate.

In a formulation that contains water, milk, fat, or oil, the percent water can be, for example, 40-50%, 50-60%, 60-70%, 70-80%, 80-90%, 90-95%, and the like. Percent of milk can be, for example, 40-50%, 50-60%, 60-70%, 70-80%, 80-90%, 90-95%, and the like. Percent of overall lipid, or of any given fat, oil, or fatty acid, can be, for example, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, and the like. Percent of any given protein or any given peptide or amino acid, can be, for example, 1-2%, 2-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, and the like.

In exclusionary embodiments, what can be excluded is a composition, and related methods, that do not comprise silicone. Also, what can be excluded is a composition and related methods, that do not comprise hyaluronic acid. Moreover, what can be excluded are compositions and related methods, that do not comprise both silicone and hyaluronic acid.

The present disclosure provides compositions, and related methods, where ratio of CBD (weight)/THC (weight) is greater than 0.02; greater than 0.05; greater than 1.0; greater than 2.0; greater than 5.0; greater than 10.0; greater than 20; greater than 50; greater than 100; greater than 200; greater than 500, greater than 1,000, greater than 2,000, greater than 5,000; greater than 10,000; greater than 20,000; greater than 50,000, and the like. In exclusionary embodiments, what can be excluded is any composition that does not meet the criteria of any of the above ratios. CBD, THC, CBN, and other compounds found in marijuana can be measured, and their ratios can be determined, by established methods (see, e.g., Poklis et al (2010) J. Anal. Toxicol. 34:516-520; Schwope et al (2011) Anal. Bioanal. Chem. 401:1273-1283). Relevant methods include solid phase extraction, liquid chromatography, and mass spectroscopy. An analysis of various cannabis plants revealed that CBD/THC occurred at a ratio of about 0.50 to about 1.6, depending on the genetics of the plant (de Meijer et al (2003) Genetics. 163:335-346), or in a ratio of 1.7-0.11, depending on the source of the plant (Pitts et al (1992) J. Pharm. Pharmacol. 44:947-951).

A ratio, or range of ratios, of CBD (weight)/THC (weight) that precludes psychoactive effects can be evaluated as follows. Psychoactive effects can be measured by tests that are sensitive to memory impairment (see, e.g., Laaris et al (2010) Neuropharmacology. 59:121-127; Wilson et al (1994) Psychiatry Res. 51:115-125), and animal learning tests, e.g., water maze test (Moore et al (2010) J. Pharmacol. Exp. Ther. 335:294-301). Tests that can be models for psychoactive effect tests include measurement of GABA release and glutamate release (Gerdman et al (2001) J. Neurophysiol. 85:468-471; Laaris et al (2010) Neuropharmacology. 59:121-127), as well as G-protein signaling tests (Balenga et al (2011) Adv. Pharmacol. 62:251-277). The present disclosure reduces, precludes, mitigates, or minimizes psychoactive effects, while maintaining or increasing effects that are one or more of anti-pain, anti-neuropathic pain, anti-inflammatory, anti-depressant, neuroprotective, or anti-neoplastic. Cannabidiol (CBD) has these effects (see, e.g., Booz (2011) Free Radic. Biol. Med. 51:1054-1061; Toth et al (2010) Molecular Pain. 6:16 (22 pages).

Silicone is an excellent barrier against water solutions and water-soluble chemicals which makes it a great combination for sport applications. Also, the mixture can be applied directly to the skin and the individual can sit in a hot bath and the moisture can still be absorbed into the body.

Current products on the market that use varying types of healing venues are amenable to using with the GJ/DJ sbm mixtures. Examples are as follows.

Therma-care wraps. Aimed to relieve soreness of everyday activities and are recommended for a wide variety of ailments from strains and sprains, to menstrual cramps. Not only do the patches stay warm for hours, but they also deliver the therapy in such a portable manner that patients can go about their normal activities while receiving the direct heat therapy.

Silicone sheeting. The main benefit of silicone sheeting compared to scar healing gels and ointments, is that silicone sheets put direct pressure on the body that is beneficial to minimizing scar appearance, and it also moves with the body for greater comfort to body movement. Silicone sheets are easier and more convenient to apply and use, because they are not greasy, do not stain, and do not stick to clothes. Silicone sheeting may act, without implying any limitation, by increased pressure, hydration, oxygen tension, and the presence of silicone in the local environment due to the application of silicone sheets. Silicone sheets produce a static electric field, resulting from friction of the silicone material, that might have an effect on wound healing as well.

Curad® scar therapy, silicone pads. Many personal care marketers are looking for ways to extend their product lines by offering new product forms. Wet wipes offer a potentially lucrative option. One way to create differentiated wipes is to vary the substrate used and its corresponding lotion. From consumers' perspective, a key component in differentiation is a pleasant skin feel with good cleansing properties. This means, formulators must consider enhances sensory profiles, including softness, reduced residue and a variety of other characteristics. These desired properties suggest greater opportunity for the use of silicones in wet wipe applications. Although silicones are widely used in personal care products, greater penetration in the wet wipes market can help formulators achieve their goals of innovative and high performance wipes.

The applications for this product combined with HLA, GJ/DJ sbm and/or silicone liquid comprise, in combination, the following:

(1) Disposable type wraps that would be embedded with, rolled on, or patched in some nature with a mixture of GJ, silicon, or HLA;

(2) Reusable flexible wraps that contain either pockets, sleeves, or attachments of some nature where disposable patches, beads, rings, strips, tubes, or the like, can be filled with the content and that portion of the wrap be disposed;

(3) Disposable strips, as those of a band aid that were embedded with the healing solution;

(4) Roll on devices containing the fluid that are either disposable or refillable;

(5) Lotions in various containers including pumps that are either disposable or refillable;

(6) Sprays that go directly on the skin in containers that are either disposable or refillable;

(7) Small tacky patches that are disposable;

(8) Gel form product either in disposable tubes or refillable containers;

(9) Cloth-like material that can be sprayed, wrapped on the body, that is, sport injury area;

(10) Wet wipe like product that can be placed on the skin and wrapped;

(11) Silicone beads embedded with HLA and GJ®;

(12) Barrier agents against water solutions and water-soluble chemicals;

This healing combination can be appreciated and used by persons with aches and pains associated with, rheumatoid arthritis, osteoid arthritis, broken bones, restless legs syndrome, neurological pain, neuropathic pain, muscle strains, muscle pains, pulled or strained ligaments, headaches caused by tension, and the like.

Various exemplary implementations of the present invention share and are based upon the principle that the combination of a cannabinoid containing composition and flax oil linseed oil, also known as flax seed oil, is a clear to yellowish oil obtained from the dried ripe seeds of the flax plant (Linum usitatissimum, Linaceae). The oil is obtained by cold pressing, sometimes followed by solvent extraction. Linseed oil is a mixture of various triglycerides that differ in terms of their fatty acid constituents. For linseed oil, the constituent fatty acids are of the following types:

The saturated acids palmitic acid (about 7%) and stearic acid (3.4-4.6%),

The monounsaturated oleic acid (18.5-22.6%),

The doubly unsaturated linoleic acid (14.2-17%),

The triply unsaturated omega-3 fatty acid α-linolenic acid (51.9-55.2%).

Food-grade flax seed oil is cold-pressed, obtained without solvent extraction, and marketed as edible flax seed oil. Fresh, refrigerated and unprocessed, linseed oil is used as a nutritional supplement. It contains the highest level of omega-3 fatty acids among vegetable oils, especially alpha-linolenic acid, which may be beneficial for reducing inflammation leading to atherosclerosis. Diane H. Morris. Flax Reduces Inflammation Leading to Atherosclerosis. New Flax Facts. Flax Council of Canada. Flax Council of Canada, Winnipeg, MB, Canada. The extracted linseed oil does not contain the ligand found in flax seed, Flax—A Healthy Food, Flax Council of Canada.

### DEFINITIONS

A composition of triglycerides from flax seed oil and cannabinoids derived from the cannabis plant is hereinafter also referred to as an F-C extract which is at least one of bioactive and bioavailable with respect to at least one of CB1 and CB2 receptors. In some instances at least one of said cannabinoids is CBD or THC.

A miquot for the purpose of the present invention is a profiled dose allocation of a particular form or embodiment, as customized or quasi-customized to a patient's profiled user needs map.

A pharmaceutically effective dose is defined by that threshold of efficacy by which a patient in need of treatment can receive a benefit.

### REGENTS AND SUPPLIERS

Reagents, such as emulsifiers, detergents, oils, surfactants, fatty acids, amino acids, are available (see, e.g., Sigma Aldrich, St. Louis, Miss.; DuPont Chemical Co., Wilmington, Del.; Gillco Ingredients, San Marcos, Calif.; Evonik Industries, Essen, Germany). Polymers, including silicone polymers, silicone sheets, and silicone pads, are available (see, e.g., Grace Bio-Labs, Bend, Ore.; Thunder Technologies, Rochester Hills, Minn.). The present disclosure provides composition, and related methods, that use one or more of the following emulsifiers. These emulsifiers include sorbitan trioleate, lecithin, sorbitan monostearate, triglyceryl monooleate, PEG-7 glyceryl monococoate, and polysorbate 80. Also provided is one or more emulsifiers with a hydrophilic/lipophilic balance (HLB) selected from, 0.5-1.0, 1.0-2.0, 2-3, 3-4, 4-5, 5-6, 6-7, 7-8, 8-9, 9-10, 10-11, 11-12, 12-13, 13-14, 14-15, 15-16, 16-17, or 17-18 (see, e.g., Rieger and Rhein (1997) Surfactants in Cosmetics, 2^{nd} ed., CRC Press, Boca Raton, Fla., pp. 129-132). Silicone oil and silicone liquid is available from various suppliers, for example, Du Pont Chemical Co., Sigma-Aldrich, and Dow-Corning. The disclosure provides, without limitation, silicone oil or silicone liquid, and combinations thereof, such as polydimethylsiloxane; octamethyltrisiloxane, hexamethylsiloxane, alkylmethyl silicone polyglycol, short hydroxyl-terminated dimethicone, and so on. Containers for foods and pharmaceuticals include, but are not limited to, rubber capped vials, screw top vials, gel capsules, frangible sealed vials or bottles, vials or bottles where the top or cap can be pried off, or containers requiring piercing or puncturing for opening, and the like. Compositions of the present disclosure can be stored or packaged using, for example, one or more of a disposable wrap, reusable flexible wrap, strip, roll-on apparatus, tacky patch, timed release patch, gel composition, cloth-like matrix or material, silicon beads, or barrier agents.

Carriers

Preparation of carriers, for example, of hyaluronic acid carrier, can be accomplished by a number of methods. The carrier and one or more compounds, can be thoroughly mixed using a Dounce homogenizer in an aqueous medium. Alternatively, the carrier and one or more compounds can be subjected to ultrasonication in an aqueous medium. Also, the carrier and one or more compounds can be dissolved in a solvent, such as 100% alcohol, 80% alcohol/20% water, 50% alcohol/50% water, 20% alcohol/80% water, followed by one or more of drying, by homogenization, or by sonication. Alternatively, the carrier and one or more compounds can be dissolved in a solvent that supports substantial solubilization, followed by transfer to a more aqueous solvent where solubility is maintained, and then followed by transfer to a solvent that is still more aqueous, where solubility is maintained. Homogenizers and sonicators are available (see, e.g., Fisher Scientific, Pittsburgh, Pa.). Silicon beads are available (see, e.g., Dow Corning, Midland, Mich.; Silicone Engineering, Blackburn Lancashire, UK). Filtering can be accomplished using a filter with pores that are about 0.05 mm in diameter, about 0.1 mm, about 0.2 mm, about 1.0 mm, about 2 mm, about 10 mm (1 centimeter), about 20 mm in diameter, and the like. The filter can also take the form of fibers, for example, filtering through a paper filter or through cheese cloth. Filters, cheese cloth, and other supplies are available from, for example, Grainger, Inc., Lake Forest, Ill..

The disclosure also encompasses carriers that are ingestible, such as baked good, dried fruit bar, confectionary, brownies, cookies, muffins, rice snacks such as Rice Krispies® snacks, candies, beverages, such as coffee, tea, or fruit drink or blended fruit-based drink, or a sauce or salad dressing, or a product based on butter or margarine. Also provided are carriers such as toiletries, soap, shampoo, lip balm, and the like.

Those of ordinary skill in the art will recognize that to effect a cannabinoid receptor and be bioactive/bioavailable such effect may include up regulation or down regulation of the receptor via modulation of other pathways or products as well as direct receptor interactions.

The stability and low toxicity of the F-C extract makes it useful as a liquid additive for consumption and/or topical use.

The F-C extract may be combined with dietary items to be consumed as a dietary supplements, beverages, food or tonic. In some instances, the amount of F-C extract added per use is F-C extract into aliquots of at least about between 0.05 and 0.25 ml of volume.

The F-C extract may also be combined with pharmaceutically acceptable carriers to be applied as a lotion, salve, cream, rinse, soap, shampoo, conditioner, cosmetic, and analgesic. In some instances, the amount of F-C extract added per use is F-C extract into miqouts of at least about between 0.05 and 0.25 ml of volume.

The F-C extract may also treat medical conditions such as inflammation, tension, loss of appetite, nervousness, and pain. In some instances, the amount of F-C extract added per use is F-C extract into doses of at least about between 0.05 and 0.25 ml of volume.

The F-C extract may also be combined with pharmaceutically acceptable carriers to form medicines for oral, inhaled, topical, injected, ingested or suppository use. In some instances, the amount of F-C extract added per use is F-C extract into miqouts of at least about between 0.05 and 0.25 ml of volume.

At room temperatures the F-C extract should not rancidify for about 2 to about 60 days. At about 36 to 42 degrees F. the F-C extract should not rancidify for about 30 to about 200 days. At about 20 to 30 degrees F. the F-C extract should not rancidify for about 30 to about 200 days.

In at least one exemplary implementation one or more cannabinoids is combined with flax seed oil resulting in a composition containing at least one of a bioactive and bioavailable cannabinoid 2 (CB2) receptor agonist and triglycerides.

In at least one exemplary implementation one or more cannabinoids are combined with flax seed oil resulting in a composition containing at least one of a bioactive and bioavailable cannabinoid 2 (CB2) receptor antagonist and triglycerides.

In at least one exemplary implementation one or more cannabinoids are combined with flax seed oil resulting in a composition containing at least one of a bioactive and bioavailable cannabinoid 1 (CB1) receptor agonist and triglycerides.

In at least one exemplary implementation one or more cannabinoids are combined with flax seed oil resulting in a composition containing at least one of a bioactive and bioavailable cannabinoid 1 (CB1) receptor antagonist and triglycerides .

In at least one exemplary implementation one or more cannabinoids are combined with flax seed oil resulting in a composition containing at least a bioactive/bioavailable cannabinoid 1 (CB1) receptor agonist or antagonist and a bioactive/bioavailable cannabinoid 2 (CB2) receptor agonist or antagonist and triglycerides.

In at least one exemplary implementation one or more cannabinoids are combined with flax seed oil resulting in a composition containing a cannabinoid receptor agonist or antagonist and triglycerides .

In at least one exemplary implementation one or more cannabinoids are combined with flax seed oil the resulting composition attaching at least one of a cannabinoid 2 (CB2) receptor agonist or antagonist and cannabinoid 1 (CB1) receptor agonist or antagonist to the carbon chain of a triglyceride from a flax seed oil.

In at least one exemplary implementation one or more cannabinoids are combined with flax seed oil and a pharmaceutically accepted carrier, included but not limited to lotions, oils and creams, resulting in a composition containing a bioactive cannabinoid receptor agonist or antagonist and triglyceride that is absorbed by the skin.

In at least one exemplary implementation one or more cannabinoids are combined with flax seed oil and a pharmaceutically accepted carrier , included but not limited to lotions, oils and creams, resulting in a composition containing a bioactive cannabinoid receptor agonist or antagonist and triglyceride that may be absorbed by mammalian systems.

In at least one exemplary implementation one or more cannabinoids are combined with flax seed oil and a digestible carrier forming a beverage , the carrier including but not limited to at least one of dairy, fruit, yogurt, coffee, tea, water, vegetable, grains, alcohol, distilled spirits and containing a bioactive/bioavailable cannabinoid receptor agonist or antagonist wherein at least a portion of the cannabinoid receptor agonists or antagonists pass through the stomach and is absorbed in the gut.

In order to better control food quality and compatibility with respective miquots of F-C extract, batches are developed which are drug-free.

In at least one exemplary implementation one or more cannabinoids are combined with flax seed oil and a digestible carrier forming a food, the food including but not limited to at least one of grain, meat, poultry, fish, fruit, vegetable and containing a bioactive or bioavailable cannabinoid receptor agonist or antagonist wherein at least a portion of the cannabinoid receptor agonists or antagonists pass through the stomach and is absorbed in the gut.

In at least one exemplary implementation one or more cannabinoids are combined with flax seed oil forming a substantially stable liquid additive containing a bioactive or bioavailable cannabinoid receptor agonist or antagonist bound to a triglyceride. Stability at room temperature (non-refrigerated), of the liquid additive, is between about 24 hours to about 30 days and is dependant on the exposure to higher temperatures. In some instances the stability at room temperature is between about 24 hours to about 10 days. In some instances the stability at room temperature is between about 12 hours to about 5 days. In some instances the stability of the liquid additive, when refrigerated is between about 24 hours and about 90 days.

### EXAMPLE 1

Step 1: Add about one gallon of flax seed oil cold pressed preferably kosher with a 214 degrees F. burn point to a container.

Step 2: Add about 228 grams of plant materials from parts of a cannabis plant. The material should be ground up to a course particulate. The mix need not be homogenous in particulate size.

Optional Step 3: Place particulate in a divider material (such a cheese cloth) that contains the particulate and allows flow of oil into the divider and oil and cannabinoids out of the divider material.

Step 3: Place particulate in the flax oil containing container.

Over 8-12 hours cycle back and forth between heat max of 213 degrees F. and cool down to between 195-198 degrees F. as follows:

Step 4: Heat the covered container with flax oil and cannabis particulate to a maximum temperature of less than 214 degrees F. and most preferably between about 213 degrees F. and 214 degrees F. and hold temperature for about 90 to 120 minutes.

Step 5: Agitate the contents of said container for about 30 seconds to about 100 seconds.

Step 6: Lower the temperature of said container to range of 195-198 degrees F. for about 90 to 120 minutes.

Step 7: Repeat steps 4 through 6 for a time period between about 5 and 20 hours. Most preferably between 8 and 12 hours.

Step 8: Cool down said container and separate the flax oil with cannabinoids also known as the “F-C extract”.

### EXAMPLE 2

Step 1: Add about one gallon of flax seed oil cold pressed preferably kosher with a 214 degrees F. burn point to a container.

Step 2: Add about 228 grams of plant materials from parts of a cannabis plant. The material should be ground up to a course particulate. The mix need not be homogenous in particulate size.

Optional Step 3: Place particulate in a divider material (such as cheese cloth) that contains the particulate and allows flow of oil into the divider and oil and cannabinoids out of the divider material.

Step 3: Place particulate in the flax oil containing container.

Over 8-12 hours cycle back and forth between heat max of 213 degrees F. and cool down to between 195-198 degrees F. as follows:

Step 4: Heat the covered container with flax oil and cannabis particulate to a maximum temperature of less than 214 degrees F. and most preferably between about 213 degrees F. and 214 degrees F. and hold temperature for about 90 to 120 minutes.

Step 5: Agitate the contents of said container for about 30 seconds to about 100 seconds.

Step 6: Lower the temperature of said container to range of 195-198 degrees F. for about 90 to 120 minutes.

Step 7: Repeat steps 4 through 6 for a time period between about 5 and 20 hours. Most preferably between 8 and 12 hours.

Step 8: Cool down said container and separate the flax oil with cannabinoids also known as the “F-C extract.”

Step 9: Add at least one of oil of rosemary and lemon oil to the, “F-C extract.”

The F-C extract formed in a containment vessel such as a crock pot or pressurized vessel as described in examples 1 and 2. Crock pot is illustrated in US2012/0095087 (U.S. Ser. No. 13/090,400), which is incorporated herein in its entirety. US2012/0095087 shows process steps to an exemplary implementation of an F-C extract, according to the present disclosure. The container with flax oil and cannabis particulate is temperature controlled. All temperatures and times listed in the process Examples 1 and 2 are based on a pressure of near atmospheric. Increasing the pressure on the flax seed oil and cannabis mixture can be used to reduce cycle times between the higher and lower temperatures. Those of ordinary skill in the art will also recognize that pressure increase may also reduce the necessary temperatures to stress (high temperature) the triglycerides (flax seed oil carbon chains) and distress (lower temperature) the triglycerides carbon chains.

It is believed that in some instances the periods of stress, at a temperature that is below the threshold temperature for degradation of the triglycerides, reversibly alters the conformational shape of the carbon chain thereby promoting attachment of one or more cannabinoids thereto.

In some exemplary implementations the F-C extract contains cannabinoids bound to a triglyceride derived from the flax seed oil. In some instances the cannabinoids bound are at least one of a CB1 and CB2 receptor modulator. In some instances, the F-C extract is suitable for ingestion and ingestion is via a liquid with a carrier that may include fruit juices, vegetable juices, water, cream, milk, yogurt, wine, distilled sprits, beer, coffee, tea, herbal teas, and carbonation.

In some exemplary implementations the F-C may be combined with a pharmaceutically acceptable carrier including but not limited to sodium cromoglycate, bronchodilators and glucocorticosteroids such as those used in pressurized metered-dose inhaler (pMDI) for inhalation. Said F-C extract may also be delivered for inhalation via wet nebulizers may be subdivided into jet and ultrasonic models (See, Inhalation Devices and Propellants(1999) 161:S44-S50 (11 Suppl.) Canadian Medical Association).

### EXAMPLE 3

Combining the F-C extract with topical carriers is embodied in multiple forms, and has achieved current medical success. Both a massage oil and a pain-mitigating cream have been developed and made available to properly credentialed users under compassionate use legislation. (See, for example, the GJ® brand of rub and massage oil available from Premium Organic Treatments, Anaheim, Calif. 92805.) Likewise, a lip balm has been developed and each of these proprietary formulations, as explained herein, and claimed below leverage the proprietary extracting system which is driven by the cannabinoid profiling which is done by independent and highly credible testing laboratories.

Likewise, the GANJA DERM line of sustainable cosmetics was evolved to generate consumer knowledge of an interest in the base cosmetological, supplement-based, organic, vegan and related brand identities, for cosmetics. (Available from OMG Outkast Marketing entities of Orange County, Calif., 92677.)

The present disclosure provides agents that enhance emulsification of hemp oil, and other compositions. Useful combinations of GJ with agents that promote emulsifying, or that promote solubilization, include the following:

GJ+glycerine/isopropanol (IPA) mixture. This exhibits phase separates/insoluble.

GJ+0.75% hyaluronic acid solution (aqueous)+polysorbate 20. This exhibits phase separates/insoluble.

GJ+propylene glycol. This exhibits phase separates/insoluble.

GJ+polyethylene glycol 400—shows some solubility but also shows a high concentration phase separation. This exhibits moderately soluble at limited quantity.

GJ in isopropanol (IPA). This exhibits fair to good solubility.

GJ+polydimethylsiloxane. This exhibits 10 cst viscosity. This shows some emulsion formation.

Essentially, as further developed throughout this document and claimed hereafter, the sustainable nature of the instant teachings drives use of the entire plant for purposes of generating extracts. Per examples one and two above, an F-C extract is readily combinable with proprietary compounds, mixtures, admixtures and related collections of desired ingredients. Those skilled in the art readily understand how to compound, entabulate, mix, and/or otherwise combine the instant teachings with a plurality of vehicles for delivery to humans and/or other mammals in need of the same.

Similarly, cannabinoid profiling enables formulators to array and establish preferred ratios for THC, CBD and CBN, inter alia. By having numerous mechanisms to store and track such data, those skilled in the art can and do have the ability to formulate various products, derived from this sustainable base for us by at least two groups having needs for medicines based upon cannabis—namely, those who desire to have psychoactive effects and those who do not. It is further respectfully proposed that among the proprietary aspects of the instant teachings are psychoactive-free formulations which remain safe and effective and modulate pain, inflammation, muscle issues, insomnia and the multiplicity of other ailments those having needs for the products of the present invention seek.

### EXAMPLE 4

F-C extracts as proposed above were combined with ingestible carriers, namely fruit juice based smoothies having desired levels of natural fruit, sugars and fats (for example). Patients needing anti-inflammatory, pain-modulating and anti-distonic support were provided input on construction of appropriate miquots, namely unit-dose administration suggestions which addressed their enumerated health concerns. Smoothies have base-flavors, stemming from Vaccinum vites idea (blueberry) or any major fruit group were proposed and provided in transportable unit-dose suspensions. Patients' data on usage history, preferred and desired medically prognosed response were stored in a database and cannabinoid profiles for various F-C extracts reviewed and optimized products selected. Chronically afflicted sufferers of amytrophic lateral sclerosis, multiple sclerosis, and advanced neuropathic degeneration (from Diabetes mellitus and Parkinson's-like disease etiologies) reported no adverse events and returned for multiple subsequent visits.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the invention both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementations, a method or process implementations, or even merely a variation of any element of these.

Particularly, it should be understood that as the disclosure relates to elements of the invention, the words for each element may be expressed by equivalent apparatus terms or method terms - even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular exemplary implementations, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative exemplary implementations.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

What is claimed is:

1. A process for producing a cannabinoid and triglyceride composition, the process comprising:
adding at least about one gallon of cold pressed flax seed oil having with a 214 degrees F. burn point to a container which can be covered;
adding at least about 228 grams of ground up plant materials per about one gallon from material derived from cannabis plants;
covering for between about 8 and up to 12 hours the container and cycling back and forth between a maximum heat of less that 214 degrees F. and a minimum heat of between at least about 195-198 degrees F. while mechanically agitating and alternating between maximum and minimum heat;
cooling a resulting admixture down to room temperature;
separating the plant material from the liquid wherein the liquid forms a flax seed oil and cannabinoids extract (F-C extract); and
combining the same with one or both of a silicone liquid carrier and hyaluronic acid (HLA) carrier.

2. The process of claim 1, wherein
the cycle times are between 90 and 120 minutes for each maximum heat and each minimum heat cycle; and,
the duration of the mechanical agitation is between at least about 30 and about 148 seconds.

3. The process of claim 1, further comprising the step of:
placing said plant material within a filtering divider material which both contains the plant material and reduces the flow of plant materials out of the filtering divider material.

4. The process of claim 1, wherein the cannabis plants further comprise cannabidiol (CBD) and delta(9)-tetrahydrocannabinol (THC), and wherein the ratio of [cannabidiol (CBD) (weight)]/[delta(9)-tetrahydrocannabinol (THC) (weight)] in the F-C extract is greater than 20/1 or is greater than 30/1.

5. A composition prepared by the process of claim 1, further comprising separating aliquots of the combined F-C extract and carrier into single doses by volume, and placing each aliquot into a separate container.

6. A product, produced by the process of claim 4.

7. A composition prepared by the process of claim 1 further comprising one or more of silicone beads, an inhalable carrier, hyaluronic acid (HLA), a fatty acid, and a fatty acid derivative.

8. A composition prepared by the process of claim 1, further comprising ingestible carrier, selected from the group a beverage, baked good, sauce, and a dressing.

9. A composition prepared by the process of claim 1, that further comprises at least one of a cosmetic or a lotion.

10. A composition prepared by the process of claim 1, comprising in combination, a topical carrier that contains:
water;
goat's milk;
Hellanthus annus oil;
soya glycine;
theobroma cacao seed butter;
butyospernum;
park fruit;
vegetable glycerin;
glycol copolymer;
isopropyl myristate;
monostearate;
acrylamide;
octyldodecanol;
butylcarbamate;
palmitate PEG-320;
idopropanol;
polysorbate;
ceterate-14;
aloe vera extract;
glyceril stearate;
carbopol;
essential oils,
hyaluronic acid, and
silicone oil.

11. A composition prepared by the method of claim 1, wherein the ratio of cannabidiol (CBD) to delta(9)-tetrahydrocannabinol (THC) is reduced, when compared to the ratio of cannabidiol (CBD) to delta(9)-tetrahydrocannabinol (THC) in the non-processed ground up plant materials.

12. The composition of claim 10, wherein the ratio of cannabidiol (CBD) to delta(9)-tetrahydrocannabinol (THC) that is reduced, results in substantially de minimus or no psychoactive effects, when compared to those produced by a composition that is the non-processed ground up plant materials.

13. A method for administering a substitute for medicinal marijuana to mammals, wherein the substitute for medical marijuana is a substance that comprises a composition prepared by the method of claim 1, the method comprising administering to a patient a substance that comprises the composition prepared by the method of claim 1, along with avocado oil.

14. A composition configured for topical application, comprising cannabidiol (CBD), hyaluronic acid, water, at least one fatty acid or fatty acid derivative, wherein the composition is a clear solution or is an emulsion when stored at 23 degrees C., and wherein the composition does not form a biphasic solution when stored at 23 degrees C.

15. The composition of claim 14, wherein the at least one fatty acid or fatty acid derivative that comprises isopropylmyristate, stearate, palmitic acid, lecithin, olive oil, or flax seed oil.

16. The composition of claim 14, wherein the ratio (weight/weight) of cannabidiol (CBD) to delta(9)-tetrahydrocannabinol (THC), is greater than 10.0/1.0, or is greater than 30.0/1.0.

17. A composition configured for topical administration, wherein the composition is derived from cannabis plants that contain cannabidiol (CBD) and delta(9)-tetrahydrocannabinol (THC), wherein the ratio (weight/weight) of cannabidiol (CBD) to delta(9)-tetrahydrocannabinol (THC) in the composition is greater than 20/1, or is greater than 30/1, and wherein the composition comprises one or both of a silicone liquid and hyaluronic acid (HLA) carrier.

18. The composition of claim 17, further comprising at least one fatty acid or fatty acid derivative that comprises isopropylmyristate, stearate, palmitic acid, lecithin, olive oil, or flax seed oil.

19. The composition of claim 17 that comprises silicone liquid and does not comprise hyaluronic acid.

20. The composition of claim 18, wherein the same does not deleteriously impact federal legal schemes or bone fide THC-based drug-testing regimes.
